(19) United States
(12) Patent Application Publication
(10) Pub. No.: US 2023/0348851 A1
(43) Pub. Date: Nov. 2, 2023

(54) BIOCOMPATIBLE SCAFFOLDS FOR CULTURING POST NATAL PROGENITOR CELLS

(71) Applicants: REGENESYS BVBA, Heverlee (BE); ANTLERON NV, Leuven (Heverlee) (BE)

(72) Inventors: Bart VAES, Heverlee (BE); Jan SCHROOTEN, Leuven (BE); Maarten SONNAERT, Wilsele (BE); Michelle STAKENBORG, Blanden (BE)

(73) Assignees: REGENESYS BVBA, Heverlee (BE); ANTLERON NV, Leuven (Heverlee) (BE)

(21) Appl. No.: 17/909,584

(22) PCT Filed: Mar. 5, 2021
(86) PCT No.: PCT/IB2021/051874

§ 371 Date: Sep. 6, 2022

Related U.S. Application Data
(60) Provisional application No. 62/986,080, filed on Mar. 6, 2020.

(51) Int. Cl. C12N 5/074

(52) U.S. Cl. CPC C12N 5/0607; C12N 2513/00; C12N 2533/90

(57) ABSTRACT

The present invention provides compositions and methods for culturing cells and preparing sheets and three-dimensional arrangements of cells that can be used for tissue repair. The invention also relates to methods of treatment using the compositions.

### RELATED APPLICATION

This application claims priority to U.S. Provisional Patent Application No. 62/986,080, filed Mar. 6, 2020.

### BACKGROUND OF THE INVENTION

### Field of the Invention

The present invention relates to specialized scaffolds onto which cells can be seeded and cultured to be used for tissue repair and tissue regeneration. The scaffolds are prepared by any method known in the art, for example by three-dimensional printing, forming gels, and/or by electrospinning fibers into a desired conformation, and are particularly suited for seeding of cells thereon, as a result of extracellular matrix (ECM) having been deposited on the surface of the scaffold. Post-natal progenitor cells (PNPC) can be used for the ECM deposition, and the PNPCs can optionally be removed and the ECM-coated scaffold can be stored for future reseeding with the PNPCs or other cells.

### Related Art

Presented below is background information on certain aspects of the present invention as they may relate to technical features referred to in the detailed description, but not necessarily described in detail. That is, certain components of the present invention may be described in greater detail in the materials discussed below. The discussion below should not be construed as an admission as to the relevance of the information to the claimed invention or the prior art effect of the material described.

Tissue engineering (TE) has made tremendous efforts to develop advanced technologies for wound care (Hu et al. 2014; Volk et al. 2013). For instance, TE has focused on the development of ECM-biomimetic materials that can potentially enhance tissue repair and stimulate regeneration. However, the identification of appropriate biomaterials and the fine balance in the quantity and quality of ECM proteins remain important considerations in the field (Volk et al. 2013). An alternative approach is to use biomaterials of cell-derived constituents.

Regenerative medicine represents a new paradigm to resolve unmet medical needs by translating fundamental knowledge from biomedicine into novel treatment strategies to augment, repair, replace or regenerate tissue.

The ECM is a major determinant in the balance between repair and regeneration in wound healing and tissue repair. Therefore, numerous ECM-like materials have been developed that can function in wound support, enhance tissue repair and regeneration or function as a cell delivery system. Biocompatibility, full incorporation into the recipient tissue and stimulation of regeneration are the most important characteristics of these next-generation matrices. However, many prior art matrix products have limitations. For instance, collagen scaffolds have high biocompatibility and are readily absorbed by the body, but the majority of these scaffolds do not comprise the typical ratio of collagen I/III in the wound environment that characterizes for instance wound healing without a scar in fetal dermis (Hu et al. 2014).

The use of coated fibers for the three dimensional culture of cells has also been described. In U.S. Pat. No. 9,766,228 (“the '228 patent”; incorporated by reference herein in its entirety for all purposes), neural cells were cultures on coated, electrospun fibers. Fibers disclosed included polystyrene (PS), poly acrylo nitrile (PAN), poly carbonate (PC), polyvinylpyrrolidone, polybutadiene (PVP), polyvinyl butyral (PVB), poly vinyl chloride (PVC), poly vinyl methyl ether (PVME), poly lacticco-glycolic acid (PLGA), poly(l-lactic acid), polyester, polycaprolactone (PCL), poly ethylene oxide (PEO), polyaniline (PANI), polyfluorenes, polypyrroles (PPY), poly ethylenedioxythiophene (PEDOT) polyether-based polyurethane or mixtures thereof. The '228 patent disclosed fibers of 900-1500 nm, preferably 1000-1400 nm and more preferably 1100-1300 nm for the cultivation of astrocytes, wherein the porosity of the electrospun fibers (air to fiber volume) was 60-95%, alternatively 65-75% or 70-90%. In another embodiment, the fibers had a diameter of 100-900 nm, more preferably about 200-800 nm, and most preferably 350-500 nm for the cultivation of neurons. The fibers were spun to a thickness of 200 micrometers or less and were optionally coated with a bio-active substance such as collagen I, poly-D-lysine, poly-L-ornithine and laminin.

To overcome current shortcomings in tissue engineering for the treatment of diseases and conditions that require tissue regeneration, including wound healing, the inventors have developed compositions that combine tissue engineering and regenerative medicine approaches. The inventors have surprisingly found that the controlled bioprocessing of PNPCs towards an ECM biomaterial that can be applied as a medical device solves the above technical problem regarding the lack of controlled and consistent adaptability of the geometry, composition and constitutive properties of artificial tissues. Therefore, the present invention relates to an advanced therapy medicinal product (ATMP) when combined with cells and/or other biologics to enhance regeneration.

### SUMMARY OF THE INVENTION

The following brief summary is not intended to include all features and aspects of the present invention, nor does it imply that the invention must include all features and aspects discussed in this summary.

The present invention provides a composition that is particularly well suited for administering to a patient to allow for tissue repair, wherein the composition comprises a culture of cells on a scaffold.

The present invention comprises, in certain aspects, a culture of PNPCs on a scaffold.

In one aspect, the scaffold that supports the PNPCs is a scaffold of fibers. The scaffold of fibers may be essentially two-dimensional, or alternatively may be of a thickness of structure considered to be three-dimensional.

In yet another aspect, the PNPCs are cultured on a 3D printed scaffold.

In another aspect, the invention provides a culture of PNPCs on a scaffold onto which extracellular matrix has been deposited.

In another aspect, the invention provides a decellularized scaffold onto which extracellular matrix has been deposited.

In another aspect, the invention provides a culture of cells on deposited extracellular matrix, wherein a scaffold is used for the deposition of ECM but then the scaffold itself is removed.

Another aspect of the invention is a method of making or culturing the composition or culture of PNPCs on the scaffold.

Another aspect of the invention is a method of producing a construct comprising a scaffold on which PNPCs are cultured.

A further aspect of the invention is a method for treating a disease or condition in a patient by administering the composition or culture of PNPCs on an ECM-coated scaffold to a patient in need thereof.

The features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### BRIEF DESCRIPTION OF THE DRAWINGS

FIG. 1 is a general schematic of how PNPC compositions of the present invention may be prepared. A three-dimensional scaffold is chosen, and PNPC, such as multipotent adult progenitor cells (MAPC), are seeded onto the scaffold, the cells deposit extracellular matrix (ECM) on the scaffold, the scaffold is optionally treated to decellularize the cells, leaving the scaffold with the ECM from the previously seeded multipotent adult progenitor cells, and then new cells (multipotent adult progenitor cells or other cells) are then reseeded onto the scaffold.

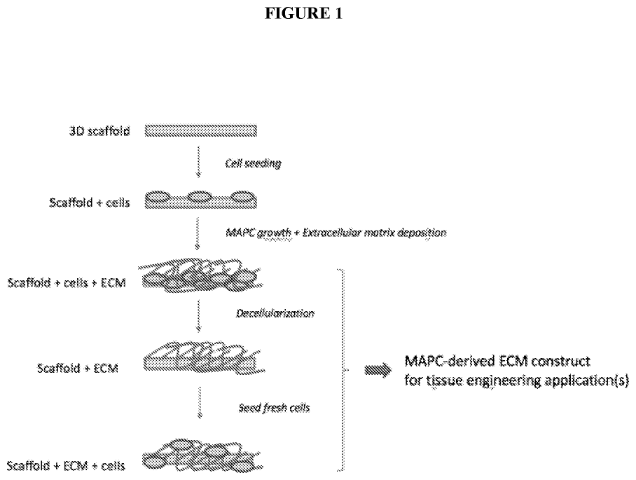

FIG. 2 is a more detailed schematic of how compositions of the present invention may be prepared. A three-dimensional scaffold is chosen and may be functionalized, such as by plasma activation, PNPCs, such as multipotent adult progenitor cells, are seeded onto the scaffold and the cells deposit extracellular matrix (ECM) on the scaffold. The cells/ECM/scaffold are then tested for collagen (PicroSirius Red staining) and for glucose consumption and lactate production (indicating cell growth). The cells may then be decellularized and the scaffold/ECM can be stored at 2-4° C. Alternatively (or subsequently), the scaffold/ECM is reseeded with MAPC or other cells and those scaffolds can be further characterized, and/or cryopreserved and/or used for animal studies and/or treatment.

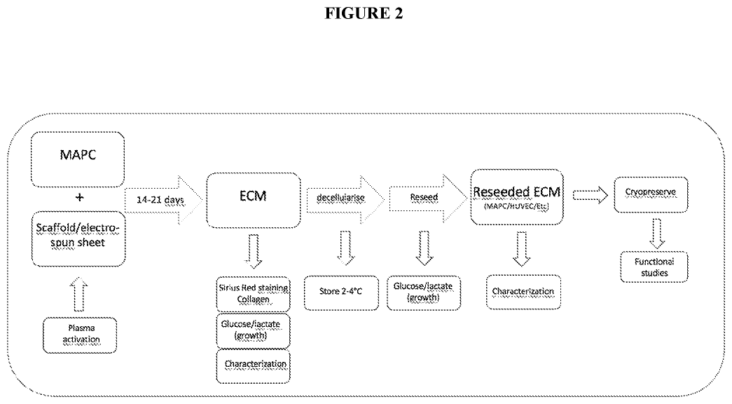

FIG. 3 shows various combinations of electrospinning materials, varying the material (PCL vs. PLA), diameter (1 μm vs. 10 μm), roughness (rough vs. smooth), orientation (random vs. semi-aligned) and temperature (ambient vs. low temperature electrospinning [LTE]). Larger fiber diameter may better support the growth and ECM production of MAPC on electrospun material.

FIG. 4A shows growth of MAPC cells on sheets by detection of glucose consumption and FIG. 4B shows lactate production. Glucose and lactate were determined by collecting spent medium and analyzed on a LaboTRACE (TRACE Analytics). The value was calculated to represent as production in mg per hour.

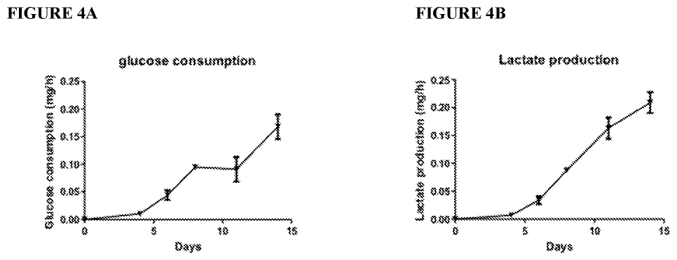

FIG. 5A shows production (nanograms/hour) of fibronectin (FN) and FIG. 5B shows pro-collagen (PIP) in spent medium at various timepoints during ECM production by MAPCs. Protein concentrations of PIP collagen and Fibronectin were determined in spent medium using AlphaLISA technology (Perkin Elmer). Protein production rates were calculated as ng per hour.

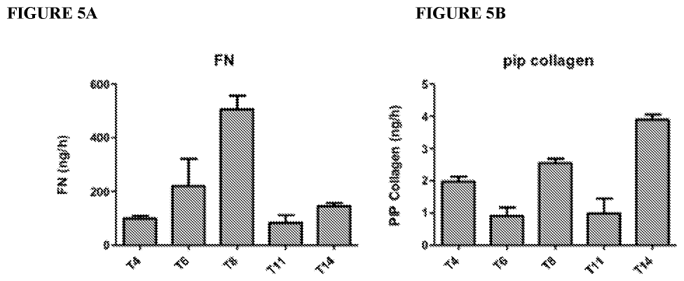

FIG. 6A shows sheets put in 12 well plates and stained with PicroSirius Red after 14 days of ECM deposition by MAPCs. FIG. 6B shows quantification of collagen detected with PicroSirius Red. To visualize matrix deposition, sheets were stained with PicroSirius red. Quantification of collagen deposition was done by extracting picrosirius red with extraction buffer of MeOH:NaOH (0.2 M), after which absorbance was measured using a plate reader.

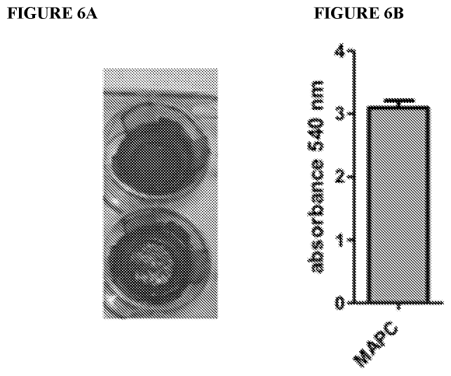

FIG. 7A shows expression of MAPC marker INSC; FIG. 7B shows expression of MAPC marker PTGS1; FIG. 7C shows expression of MAPC marker ANGPTL4. MAPC markers in cells after 14 days of matrix deposition. The MAPC markers are considered positive when they exceed a minimum expression threshold. Marker expression data were generated using RNA extracted from cells after 14 days of matrix deposition. The RNA was converted into cDNA and subsequently expression values were determined by means of qPCR using 5′ hydrolysis probes. The gene of interest is compared to a reference gene (ATP5B) with a constant expression over different samples. In this example, MAPCs express INSC at a level greater than 0.05 relative to ATP5B; express PTGS1 at a level greater than 0.05 relative to ATP5B; and express ANGPTL4 at a level greater than 0.2 relative to ATP5B, as determined by converting RNA into cDNA and quantifying with qPCR using 5′ hydrolysis probes.

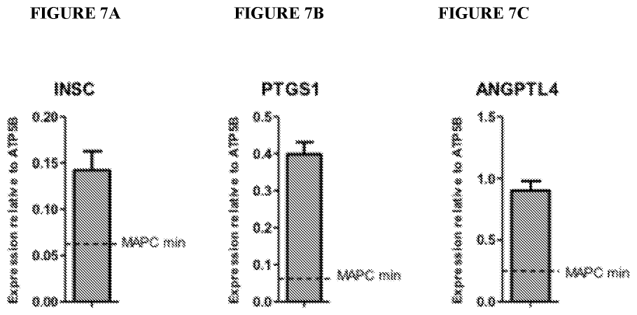

### DETAILED DESCRIPTION OF THE INVENTION

### Definitions

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well-known and commonly used in the art. Certain experimental techniques, not specifically defined, are generally performed according to conventional methods well known in the art and as described in various general and more specific references that are cited and discussed throughout the present specification. For purposes of the clarity, following terms are defined below.

“A” or “an” means herein one or more than one; at least one. Where the plural form is used herein, it generally includes the singular.

The term “bio-active substrate” refers to for example polycaprolactone (PCL), polylactide (PLA) and other similar compounds described herein, as well as their functional peptide groups.

A “cell bank” is industry nomenclature for cells that have been grown and stored for future use. Cells may be stored in aliquots. They can be used directly out of storage or may be expanded after storage. This is a convenience so that there are “off the shelf” cells available for administration. The cells may already be stored in a pharmaceutically-acceptable excipient so they may be directly administered or they may be mixed with an appropriate excipient when they are released from storage. Cells may be frozen or otherwise stored in a form to preserve viability. In one embodiment of the invention, cell banks are created in which the cells have been selected for enhanced potency to achieve the effects described in this application. Following release from storage, and prior to administration, it may be preferable to again assay the cells for potency. This can be done using any of the assays, direct or indirect, described in this application or otherwise known in the art. Then cells having the desired potency can then be administered. Banks can be made using autologous cells (derived from the organ donor or recipient). Or banks can contain cells for allogeneic uses.

“Co-administer” with respect to this invention means to administer together two or more agents.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of” and similar phrases using words of the root “comprise” are used herein as synonyms of “comprising” and have the same meaning.

“Comprised of” is a synonym of “comprising” (see above).

The term “construct” refers to the combination of elements used for the treatment of tissue damage, including at least two of scaffold, ECM or cells, wherein the cells may be PNPCs.

The term “container” as used herein means any container suitable for culturing cells in for example a plate, dish or tube.

“Conditioned cell culture medium” is a term well-known in the art and refers to medium in which cells have been grown. As used herein, the phrase means that cells are grown in the medium for a sufficient time to secrete factors that are effective for cell growth of a specified type for which the medium is being conditions.

The term “contact”, when used in relation to a cell and the scaffold to be transplanted, can mean that, upon exposure to the scaffold, the cell physically touches the scaffold and/or the ECM coated on the scaffold.

“Decrease” and “decreasing” and similar terms are used herein generally to mean to lessen in amount or value or effect, as by comparison to another amount, value or effect. A decrease in a particular value or effect may include any significant percentage decrease, for example, at least 5%, at least 10%, at least 20%, at least 30%, at least 50%, at least 75% or at least 90%.

“Effective amount” generally means an amount which achieves the specific desired effects described in this application. For example, an effective amount is an amount sufficient to effectuate a beneficial or desired clinical result. Within the context of this invention generally the desired effect is a clinical improvement compensating for the tissue damage present in a subject. The effective amounts can be provided all at once in a single administration or in fractional amounts that provide the effective amount in several administrations. The precise determination of what would be considered an effective amount may be based on factors individual to each subject, including the severity of the disease/deficiency, health of the patient, age, etc. One skilled in the art will be able to determine the effective amount based on these considerations which are routine in the art. As used herein, “effective dose” means the same as “effective amount.”

Accordingly, an “effective amount” of cells are an amount in which the clinical symptoms of the subject are improved. And an effective amount of cells would be that which is sufficient to produce a tissue graft that provide that improved clinical outcome.

“Effective route” generally means a route which provides for delivery of an agent to a desired compartment, system, or location. For example, an effective route is one through which an agent can be administered to provide at the desired site of action an amount of the agent sufficient to effectuate a beneficial or desired clinical result (in the present case, effective transplantation).

The term “exogenous”, when used in relation to a cell, generally refers to a cell that is external to the subject and which has been exposed to (e.g., contacted with) the scaffold that is intended for transplantation by an effective route. An exogenous cell may be from the same subject or from a different subject. In one embodiment, exogenous cells can include cells that have been harvested from a subject, isolated, expanded ex vivo, and then exposed to, or reseeded on the scaffold intended for transplantation by an effective route.

The term “expose” can include the act of contacting one or more cells with the scaffold intended for transplantation or contacting the damaged tissue with the scaffold containing the cells.

The term “fiber” used herein refers to a fiber made of a non-cytotoxic polymer which may be comprised of but is not limited to polycaprolactone or polylactide fibers, or any other non-cytotoxic fiber described herein.

The term “aligned fibers” as used herein refers to a fiber scaffold that consists of one or more fibers that are oriented in parallel to each other during the electrospinning process.

The term “biocompatible fiber” refers to fibers as described within this description, examples and claims, which are comprised of a material that is non-cytotoxic.

The term “coated fibers” refers to fibers as described above, the coat may be, for example, poly-L-ornithine+laminin or poly-D-lysine.

The term “coated fiber scaffold for three dimensional PNPC culture” as used herein refers to a structure comprised of one or more random oriented fibers, optionally coated with bio-active substrates as described herein, creating an environment supporting the growth of PNPCs in a three dimensional fashion.

The term “randomly oriented fibers” as used herein refers to a fiber scaffold consisting of electrospun fibers that have not been actively aligned or that do not follow any designed pattern of orientation to each other.

Use of the term “includes” is not intended to be limiting.

“Increase” or “increasing” means to induce a biological event entirely or to increase the degree of the event. For example, increasing may include a measurement which is at least 5%, 10%, 20%, 30%, 50%, 75%, or 90% more than a measured level prior to inducing the biological event.

The term “isolated” refers to a cell or cells which are not associated with one or more cells or one or more cellular components that are associated with the cell or cells in vivo. An “enriched population” means a relative increase in numbers of a desired cell relative to one or more other cell types in vivo or in primary culture.

However, as used herein, the term “isolated” does not indicate the presence of only the cells of the invention. Rather, the term “isolated” indicates that the cells of the invention are removed from their natural tissue environment and are present at a higher concentration as compared to the normal tissue environment. Accordingly, an “isolated” cell population may further include cell types in addition to the cells of the invention cells and may include additional tissue components. This also can be expressed in terms of cell doublings, for example. A cell may have undergone 10, 20, 30, 40 or more doublings in vitro or ex vivo so that it is enriched compared to its original numbers in vivo or in its original tissue environment (e.g., bone marrow, peripheral blood, placenta, umbilical cord, umbilical cord blood, etc.).

“MAPC” is an acronym for “multipotent adult progenitor cell.” As used herein, “MAPC” refers to cells having two or more of the following characteristics: telomerase activity with extended replicative capacity (e.g., 40 cell doublings or more), normal karyotype, CD34³¹ , CD45⁻, CXCL5⁺, PTGSI⁺, ANGPTL4⁺, low or no HLAII, CD90⁺, CD49c⁺, and may be induced in vitro to differentiate into osteoblasts, adipocytes, and chondrocytes. MAPCs have also been reported to have the ability to differentiate into cells of the ectodermal germ layer and cells of the endodermal germ layer (Jiang et al., Nature 2002, 18:41-49). “Low or no” expression may include expression that is about 30%, 25%, 20%, 15%, 10% or 5% of a measurement considered to indicate positive expression. According to the present invention, whenever a composition or method includes a post natal progenitor cell (PNPC), it is understood that in any and all of those compositions and/or methods MAPCs could be chosen as the post natal progenitor cell (PNPC). Thus, MAPC is a specific embodiment of a post natal progenitor cell (PNPC), and, accordingly, is relevant to all of the compositions and methods described in this application.

“May” as used herein means the same as “optionally” and even where it is not stated, as used herein, “may” includes also that it “may not”. That is, a statement that something may be, means as well that it also may not be. That is, as used herein, “may” includes “may not”, explicitly, and applicant reserves the right to claim subject matter accordance therewith. For instance, as used herein, the statement that PNPCs may be administered with other agents, also means that PNPCs may be administered without any other agents. For another example, as used herein the statement that PNPCs may be genetically engineered also means that PNPCs may be not genetically engineered.

“MultiStem®” is the trade name for a cell preparation based on the MAPCs of U.S. Pat. No. 7,015,037. MultiStem® can be prepared according to cell culture methods described below. Gene expression and differentiation potential as described in paragraph

MultiStem® is highly expandable, karyotypically normal, not tumorigenic, not transformed, and does not form teratomas in vivo.

“Optionally” as used herein means much the same as “may”. The statement that X optionally includes A as used herein includes both X includes A and X does not include A.

“Pharmaceutically-acceptable carrier” is any pharmaceutically-acceptable medium for the cells and/or scaffold used in the present invention. Such a medium may retain isotonicity, cell metabolism, pH, and the like. It is compatible with administration to a subject and can be used, therefore, for scaffold and/or cell delivery and treatment.

The term “plastic material” may be used to refer to the fibers described herein, and refers to polymers including polystyrene (PS), polyacrylonitrile (PAN), polycarbonate (PC), polyvinylpyrrolidone (PVP), polybutadiene, polyvinylbutyral (PVB), polyvinyl chloride (PVC), polyvinyl methyl ether (PVME), poly lacticco-glycolic acid (PLGA), poly(1-lactic acid) (PLA), polyester, polycaprolactone (PCL), poly ethylene oxide (PEO), polyaniline (PANI), polyfluorenes, polypyrroles (PPY), poly ethylene dioxythiophene (PEDOT) polyurethane (PU), polyphosphazenes, poly(propylene carbonate), poly(vinyl alcohol) (PVA), poly(3-hydroxybutyrate-co-3-hydroxyvalerate) (PHBV), poly acrylo nitrile (PAN), poly vinyl methyl ether (PVME) or a mixture of two or more of those.

A “post-natal progenitor cell” is a progenitor cell that can form a progeny cell that is more highly differentiated than the progenitor cell. These cells have extended replicative capacity in culture (>40 doublings), telomerase activity, normal karyotype, not tumorigenic, and secrete ECM proteins. The term “progenitor” does not limit these cells to a particular lineage.

The term “reduce” as used herein means to prevent as well as decrease. In the context of treatment, to “reduce” is to either prevent or ameliorate the deficiency. This includes causes or symptoms of tissue damage. For example, reducing may include a measurement which is at least 5%, 10%, 20%, 30%, 50%, 75%, 90% or 100% less than what is measured prior to treatment.

“Selecting” a cell with a desired level of potency can mean identifying (as by assay), isolating, and expanding a cell. This could create a population that has a higher potency than the parent cell population from which the cell was isolated. The “parent” cell population refers to the parent cells from which the selected cells divided. “Parent” refers to an actual P1→F1 relationship (i.e., a progeny cell). So if cell X is isolated from a mixed population of cells X and Y, in which X is an expressor and Y is not, one would not classify a mere isolate of X as having enhanced expression. But, if a progeny cell of X is a higher expressor, one would classify the progeny cell as having enhanced expression.

To select a cell that achieves the desired effect would include both an assay to determine if the cells achieve the desired effect and would also include obtaining those cells. The cell may naturally achieve the desired effect in that the effect is not achieved by an exogenous transgene/DNA. But an effective cell may be improved by being incubated with or exposed to an agent that increases the effect. The cell population from which the effective cell is selected may not be known to have the potency prior to conducting the assay. The cell may not be known to achieve the desired effect prior to conducting the assay. As an effect could depend on gene expression and/or secretion, one could also select on the basis of one or more of the genes that cause the effect.

Selection could be from cells in a tissue. For example, in this case, cells would be isolated from a desired tissue, expanded in culture, selected for achieving the desired effect, and the selected cells further expanded.

Selection could also be from cells ex vivo, such as cells in culture. In this case, one or more of the cells in culture would be assayed for achieving the desired effect and the cells obtained that achieve the desired effect could be further expanded.

Cells could also be selected for enhanced ability to achieve the desired effect. In this case, the cell population from which the enhanced cell is obtained already has the desired effect. Enhanced effect means a higher average amount per cell than in the parent population.

The parent population from which the enhanced cell is selected may be substantially homogeneous (the same cell type). One way to obtain such an enhanced cell from this population is to create single cells or cell pools and assay those cells or cell pools to obtain clones that naturally have the enhanced (greater) effect (as opposed to treating the cells with a modulator that induces or increases the effect) and then expanding those cells that are naturally enhanced.

However, cells may be treated with one or more agents that will induce or increase the effect. Thus, substantially homogeneous populations may be treated to enhance the effect.

If the population is not substantially homogeneous, then, it is preferable that the parental cell population to be treated contains at least 100 of the desired cell type in which enhanced effect is sought, more preferably at least 1,000 of the cells, and still more preferably, at least 10,000 of the cells. Following treatment, this sub-population can be recovered from the heterogeneous population by known cell selection techniques and further expanded if desired.

Thus, desired levels of effect may be those that are higher than the levels in a given preceding population. For example, cells that are put into primary culture from a tissue and expanded and isolated by culture conditions that are not specifically designed to produce the effect may provide a parent population. Such a parent population can be treated to enhance the average effect per cell or screened for a cell or cells within the population that express greater degrees of effect without deliberate treatment. Such cells can be expanded then to provide a population with a higher (desired) expression.

“Self-renewal” of a cell refers to the ability to produce replicate daughter cells having differentiation potential that is identical to those from which they arose. A similar term used in this context is “proliferation.”

“Subject” means a vertebrate, such as a mammal, such as a human. Mammals include, but are not limited to, humans, dogs, cats, horses, cows, and pigs.

The term “substrate” as used herein refers to any surface such as, but not limited to, plastic or glass, on which the cells are seeded onto.

The term “therapeutically effective amount” refers to the amount of an agent determined to produce any therapeutic response in a mammal. For example, effective therapeutic agents may prolong the survivability of the patient, and/or inhibit overt clinical symptoms. Treatments that are therapeutically effective within the meaning of the term as used herein, include treatments that improve a subject's quality of life even if they do not improve the disease outcome per se. Such therapeutically effective amounts are readily ascertained by one of ordinary skill in the art. Thus, to “treat” means to deliver such an amount. Thus, treating can prevent or ameliorate any pathological symptoms.

In the context of the invention a therapeutically effective amount is that amount of cells that beneficially affect the tissue damage to the extent that transplantation of the scaffold/cells results in an improvement in the clinical outcome. Accordingly, the effective amounts of cells can be determined by routine empirical experimentation.

The term “therapeutically effective time” can refer to the time necessary to contact the scaffold/cells with the damaged tissue in order to allow the cells to repair, lessen or decrease the tissue damage.

A therapeutically effective time could also refer to the time required for a subject to receive the scaffold and cells and achieve an improved clinical status.

The term “therapeutically effective route” refers to the routes of administration that may be effective for achieving an improved clinical outcome. The therapeutically effective route means that the cells and scaffold would be transplanted at whatever site the cells can produce their beneficial effect. Local administration can be done by any of the effective routes that are known in the art.

In determining an appropriate amount of cells to achieve the beneficial effects is determined empirically on the basis of providing the scaffold with the ability to achieve improved tissue damage. As exemplified herein, a dose range for the composite could be from tens of thousands of cells to hundreds of millions of cells. In certain embodiments, the number of cells is about at least 50,000 cells, in one embodiment in the range of about 50,000-20 million cells. In another embodiment, the number of cells is about 100,000-1 million cells. In yet another embodiment, the number of cells is in the range of about 250,000-500,000 cells. Thus, these amounts need to be determined empirically based on the method of delivery, the severity of the illness, and the like.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

“Validate” means to confirm. In the context of the invention, one confirms that scaffold has a desired ability to beneficially affect the tissue damage to be treated. This is so that one can then use that cell with a reasonable expectation of efficacy. Accordingly, to validate means to confirm that the cells, having been originally found to have/established as having the desired activity, in fact, retain that activity. Thus, validation is a verification event in a two-event process involving the original determination and the follow-up determination. The second event is referred to herein as “validation.”

The present invention relates to compositions comprising PNPCs, wherein the cells are seeded onto a scaffold, including a scaffold of fibers or a three dimensional (3D) printed scaffold. The invention also relates to compositions comprising a scaffold onto which the PNPCs have been seeded. The invention also relates to compositions comprising a scaffold onto which the PNPCs have been seeded and on which they have deposited extracellular matrix (ECM). The scaffold may optionally be decellularized, and optionally recellularized with PNPCs or another cell type. The invention further relates to compositions comprising the scaffold, the PNPCs-generated ECM, and newly reseeded cells. The invention also relates to methods of making and using the compositions and cell cultures. In some aspects, the invention relates to methods of treatment involving tissue engineering (TE), in which the compositions of the present invention are used to repair damaged tissue, for example in wound repair, for regenerating vasculature, and for regenerating other tissues damaged by injury and/or disease.

### Description of the Invention

The present invention relates to compositions comprising a scaffold that is particularly well suited for the delivery of cells to an area of tissue damage or an area in which tissue regeneration is desired. In particular, the invention relates to a 3D printed or electrospun scaffold onto which ECM has been deposited by PNPCs.

The invention further relates to compositions comprising the scaffold, the PNPCs-generated ECM, and newly reseeded cells.

The invention also relates to methods of making and using the compositions and cell cultures. In some aspects, the invention relates to methods of treatment involving tissue engineering (TE), in which the compositions of the present invention are used to repair damaged tissue, for example in wound repair, for regenerating vasculature, and for regenerating other tissues damaged by injury and/or disease.

In one embodiment, the invention is a biomimetic extracellular matrix (ECM)-based structure, utilizing an absorbable polymer that keeps its tensile strength for several weeks. The sheet/structure may be functionalized with a combination of extracellular matrix and PNPCs to facilitate ingrowth of cells (e.g., endothelial or other) and to regulate the immune response.

In yet more detail, the present invention is described by the following items which represent preferred embodiments thereof.

1. A composition comprising post-natal progenitor cells (PNPCs) seeded onto a 3D scaffold or a scaffold of fibers.
2. A culture of PNPCs, seeded onto a 3D scaffold or a scaffold of fibers.
3. A method for treating a disease or condition in a patient, comprising administering to said patient a composition comprising PNPCs seeded onto a 3D scaffold or a scaffold of fibers.
4. A method for making a cell composition, comprising seeding progenitor cells onto a 3D scaffold or a scaffold of fibers.
5. A method for culturing cells, comprising seeding PNPCs onto a 3D scaffold or a scaffold of fibers.
6. A composition comprising PNPCs seeded onto a 3D scaffold or a scaffold of fibers onto which extracellular matrix from the cells has been deposited.
7. A culture of undifferentiated PNPCs seeded onto a 3D scaffold or a scaffold of fibers onto which extracellular matrix from the cells has been deposited.
8. A method for treating a disease or condition in a patient, comprising administering to said patient a composition comprising PNPCs seeded onto a 3D scaffold or a scaffold of fibers onto which extracellular matrix from the cells has been deposited.
9. A method for making a cell composition, comprising seeding PNPCs, onto a 3D scaffold or a scaffold of fibers and allowing the cells to deposit extracellular matrix onto the fibers.
10. A method for culturing cells, comprising seeding PNPCs onto a 3D scaffold or a scaffold of fibers onto which extracellular matrix from the cells has been deposited.
11. A biocompatible scaffold prepared by seeding PNPCs onto fibers.
12. A method for treating a disease or condition in a patient, comprising administering to said patient a biocompatible scaffold prepared by seeding PNPCs onto fibers.
13. A method for making a biocompatible scaffold, comprising seeding PNPCs onto a 3D scaffold or a scaffold of fibers.
14. A biocompatible scaffold prepared by: (a) seeding PNPCs onto a 3D scaffold or scaffold of fibers; and (b) allowing the cells to deposit extracellular matrix onto the scaffold.
15. A method for treating a disease or condition in a patient, comprising administering to said patient a biocompatible scaffold prepared by: a) seeding PNPCs onto a 3D scaffold or scaffold of fibers; and (b) allowing the cells to deposit extracellular matrix onto the scaffold.
16. A method for making a biocompatible scaffold, comprising a) seeding PNPCs onto a 3D scaffold or scaffold of fibers; and (b) allowing the cells to deposit extracellular matrix onto the scaffold.
17. A biocompatible scaffold prepared by: (a) seeding PNPCs onto a 3D scaffold or scaffold of fibers; (b) allowing the cells to deposit extracellular matrix onto the scaffold; and (c) optionally decellularizing the cells.
18. A method for making a biocompatible scaffold, comprising: (a) seeding PNPCs onto a 3D scaffold or scaffold of fibers; (b) allowing the cells to deposit extracellular matrix onto the scaffold; and (c) optionally decellularizing the cells.
19. A biocompatible scaffold prepared by: (a) seeding PNPCs onto a 3D scaffold or scaffold of fibers; (b) allowing the cells to deposit extracellular matrix onto the scaffold; (c) optionally decellularizing the cells; and (d) optionally reseeding cells onto the scaffold to produce a re-cellularized scaffold.
20. A method for treating a disease or condition in a patient, comprising administering to said patient a biocompatible scaffold prepared by: (a) seeding PNPCs onto a 3D scaffold or scaffold of fibers; (b) allowing the cells to deposit extracellular matrix onto the scaffold; (c) optionally decellularizing the cells; and (d) optionally reseeding cells onto the scaffold to produce a re-cellularized scaffold.
21. A method for making a biocompatible scaffold, comprising: (a) seeding PNPCs onto a 3D scaffold or scaffold of fibers; (b) allowing the cells to deposit extracellular matrix onto the scaffold; (c) optionally decellularizing the cells; and (d) optionally reseeding cells onto the scaffold to produce a re-cellularized scaffold.
22. The composition of any one of the foregoing items, wherein the PNPCs are not induced to differentiate.
23. The culture of any one of the foregoing items, wherein the PNPCs are not induced to differentiate.
24. The method of any one of the foregoing items, wherein the PNPCs are not induced to differentiate.
25. The biocompatible scaffold of any one of the foregoing items, wherein the PNPCs are not induced to differentiate.
26. The composition of any one of the foregoing items, wherein the PNPCs are human.
27. The culture of any one of the foregoing items, wherein the PNPCs are human.
28. The method of any one of the foregoing items, wherein the PNPCs are human.
29. The biocompatible scaffold of any one of the foregoing items, wherein the PNPCs are human.
30. The composition of any one of the foregoing items, wherein the PNPCs are non-endothelial.
31. The culture of any one of the foregoing items, wherein the PNPCs are non-endothelial.
32. The method of any one of the foregoing items, wherein the PNPCs are non-endothelial.
33. The biocompatible scaffold of any one of the foregoing items, wherein the PNPCs are non-endothelial.
34. The composition of any one of the foregoing items, wherein the PNPCs are bone marrow-derived.
35. The culture of any one of the foregoing items, wherein the PNPCs are bone marrow-derived.
36. The method of any one of the foregoing items, wherein the PNPCs are bone marrow-derived.
37. The biocompatible scaffold of any one of the foregoing items, wherein the PNPCs are bone marrow-derived.
38. The composition of any one of the foregoing items, wherein the fibers are electrospun.
39. The culture of any one of the foregoing items, wherein the fibers are electrospun.
40. The method of any one of the foregoing items, wherein the fibers are electrospun.
41. The biocompatible scaffold of any one of the foregoing items, wherein the fibers are electrospun.
42. The composition of any one of the foregoing items, wherein the fibers are randomly oriented.
43. The culture of any one of the foregoing items, wherein the fibers are randomly oriented.
44. The method of any one of the foregoing items, wherein the fibers are randomly oriented.
45. The biocompatible scaffold of any one of the foregoing items, wherein the fibers are randomly oriented.
46. The composition of any one of the foregoing items, wherein the fibers are aligned.
47. The culture of any one of the foregoing items, wherein the fibers are aligned.
48. The method of any one of the foregoing items, wherein the fibers are aligned.
49. The biocompatible scaffold of any one of the foregoing items, wherein the fibers are aligned.
50. The composition of any one of the foregoing items, wherein the fibers are cross-aligned.
51. The culture of any one of the foregoing items, wherein the fibers are cross-aligned.
52. The method of any one of the foregoing items, wherein the fibers are cross-aligned.
53. The biocompatible scaffold of any one of the foregoing items, wherein the fibers are cross-aligned.
54. The composition of any one of the foregoing items, wherein the scaffold further comprises a bio-active coating.
55. The culture of any one of the foregoing items, wherein the scaffold further comprises a bio-active coating.
56. The method of any one of the foregoing items, wherein the scaffold further comprises a bio-active coating.
57. The biocompatible scaffold of any one of the foregoing items, wherein the scaffold further comprises a bio-active coating.
58. The composition of any one of the foregoing items, wherein the fibers have a diameter of 1000-10000 nm.
59. The culture of any one of the foregoing items, wherein the fibers have a diameter of 1000-10000 nm.
60. The method of any one of the foregoing items, wherein the fibers have a diameter of 1000-10000 nm.
61. The biocompatible scaffold of any one of the foregoing items, wherein the fibers have a diameter of 1000-10000 nm.
62. The composition of any one of the foregoing items, wherein the fibers are biodegradable.
63. The culture of any one of the foregoing items, wherein the fibers are biodegradable.
64. The method of any one of the foregoing items, wherein the fibers are biodegradable.
65. The biocompatible scaffold of any one of the foregoing items, wherein the fibers are biodegradable.
66. The composition of any one of the foregoing items, wherein the fibers are natural polymers.
67. The culture of any one of the foregoing items, wherein the fibers are natural polymers.
68. The method of any one of the foregoing items, wherein the fibers are natural polymers.
69. The biocompatible scaffold of any one of the foregoing items, wherein the fibers are natural polymers.
70. The composition of any one of the foregoing items, wherein the natural polymer is alginate, cellulose, chitin, chitosan, hydroxyapatite, hyaluronic acid, starch, dextran, heparin, silk, gelatin, keratin or fibrinogen.
71. The culture of any one of the foregoing items, wherein the natural polymer is alginate, cellulose, chitin, chitosan, hydroxyapatite, hyaluronic acid, starch, dextran, heparin, silk, gelatin, keratin or fibrinogen.
72. The method of any one of the foregoing items, wherein the natural polymer is alginate, cellulose, chitin, chitosan, hydroxyapatite, hyaluronic acid, starch, dextran, heparin, silk, gelatin, keratin or fibrinogen.
73. The biocompatible scaffold of any one of the foregoing items, wherein the natural polymer is alginate, cellulose, chitin, chitosan, hydroxyapatite, hyaluronic acid, starch, dextran, heparin, silk, gelatin, keratin or fibrinogen.
74. The composition of any one of the foregoing items, wherein the fibers are synthetic polymers.
75. The culture of any one of the foregoing items, wherein the fibers are synthetic polymers.
76. The method of any one of the foregoing items, wherein the fibers are synthetic polymers.
77. The biocompatible scaffold of any one of the foregoing items, wherein the fibers are synthetic polymers.
78. The composition of any one of the foregoing items, wherein the polymers are poly(α-hydroxy acids).
79. The culture of any one of the foregoing items, wherein the polymers are poly(α-hydroxy acids).
80. The method of any one of the foregoing items, wherein the polymers are poly(α-hydroxy acids).
81. The biocompatible scaffold of any one of the foregoing items, wherein the polymers are poly(α-hydroxy acids).
82. The composition of any one of the foregoing items, wherein the poly(α-hydroxy acids) are lactic (PLA) or glycolic acids.
83. The culture of any one of the foregoing items, wherein the poly(α-hydroxy acids) are lactic (PLA) or glycolic acids.
84. The method of any one of the foregoing items, wherein the poly(α-hydroxy acids) are lactic (PLA) or glycolic acids.
85. The biocompatible scaffold of any one of the foregoing items, wherein the poly(α-hydroxy acids) are lactic (PLA) or glycolic acids.
86. The composition of any one of the foregoing items, wherein the polymer is poly(lactic acid-co-glycolic acid) (PLGA).
87. The culture of any one of the foregoing items, wherein the polymer is poly(lactic acid-co-glycolic acid) (PLGA).
88. The method of any one of the foregoing items, wherein the polymer is poly(lactic acid-co-glycolic acid) (PLGA).
89. The biocompatible scaffold of any one of the foregoing items, wherein the polymer is poly(lactic acid-co-glycolic acid) (PLGA).
90. The composition of any one of the foregoing items, wherein the poly(α-hydroxy acids) are polyhydroxy alkanoate (PHA), polydroxybutyrate (PHB), poly(3-hydroxybutyrate-co-3-hydroxyvalerate) (PHBV).
91. The culture of any one of the foregoing items, wherein the poly(α-hydroxy acids) are polyhydroxy alkanoate (PHA), polydroxybutyrate (PHB), poly(3-hydroxybutyrate-co-3-hydroxyvalerate) (PHBV).
92. The method of any one of the foregoing items, wherein the poly(α-hydroxy acids) are polyhydroxy alkanoate (PHA), polydroxybutyrate (PHB), poly(3-hydroxybutyrate-co-3-hydroxyvalerate) (PHBV).
93. The biocompatible scaffold of any one of the foregoing items, wherein the poly(α-hydroxy acids) are polyhydroxy alkanoate (PHA), polydroxybutyrate (PHB), poly(3-hydroxybutyrate-co-3-hydroxyvalerate) (PHBV).
94. The composition of any one of the foregoing items, wherein the polymer is poly(ε-caprolactone) (PCL).
95. The culture of any one of the foregoing items, wherein the polymer is poly(ε-caprolactone) (PCL).
96. The method of any one of the foregoing items, wherein the polymer is poly(ε-caprolactone) (PCL).
97. The biocompatible scaffold of any one of the foregoing items, wherein the polymer is poly(ε-caprolactone) (PCL).
98. The composition of any one of the foregoing items, wherein the polymer is polyurethane (PU), poly(ethylene oxide) or polyphosphazene.
99. The culture of any one of the foregoing items, wherein the polymer is polyurethane (PU), poly(ethylene oxide) or polyphosphazene.
100. The method of any one of the foregoing items, wherein the polymer is polyurethane (PU), poly(ethylene oxide) or polyphosphazene.
101. The biocompatible scaffold of any one of the foregoing items, wherein the polymer is polyurethane (PU), poly(ethylene oxide) or polyphosphazene.
102. The composition of any one of the foregoing items, wherein the polymer is supramolecular.
103. The culture of any one of the foregoing items, wherein the polymer is supramolecular.
104. The method of any one of the foregoing items, wherein the polymer is supramolecular.
105. The biocompatible scaffold of any one of the foregoing items, wherein the polymer is supramolecular.
106. The composition of any one of the foregoing items, wherein the fibers are multi-walled carbon nanotubes.
107. The culture of any one of the foregoing items, wherein the fibers are multi-walled carbon nanotubes.
108. The method of any one of the foregoing items, wherein the fibers are multi-walled carbon nanotubes.
109. The biocompatible scaffold of any one of the foregoing items, wherein the fibers are multi-walled carbon nanotubes.
110. The composition of any one of the foregoing items, wherein the fibers are coated to increase roughness.
111. The culture of any one of the foregoing items, wherein the fibers are coated to increase roughness.
112. The method of any one of the foregoing items, wherein the fibers are coated to increase roughness.
113. The biocompatible scaffold of any one of the foregoing items, wherein the fibers are coated to increase roughness.
114. The composition of any one of the foregoing items, wherein the fibers are coated with poly(ethylene oxide terephthalate)/poly(butylene terephthalate), oxygen and argon.
115. The culture of any one of the foregoing items, wherein the fibers are coated with poly(ethylene oxide terephthalate)/poly(butylene terephthalate), oxygen and argon.
116. The method of any one of the foregoing items, wherein the fibers are coated with poly(ethylene oxide terephthalate)/poly(butylene terephthalate), oxygen and argon.
117. The biocompatible scaffold of any one of the foregoing items, wherein the fibers are coated with poly(ethylene oxide terephthalate)/poly(butylene terephthalate), oxygen and argon.
118. The composition of any one of the foregoing items, wherein the fibers have a roughness value (Rₐ) of about 10 nm-about 100 μm.
119. The culture of any one of the foregoing items, wherein the fibers have a roughness value (Rₐ) of about 100 nm-about 500 μm.
120. The method of any one of the foregoing items, wherein the fibers have a roughness value (Rₐ) of about 1 μm-about 10.
121. The biocompatible scaffold of any one of the foregoing items, wherein the fibers have a roughness value (Rₐ) of μm about 2 μm-about 5 μm.
122. The composition of any one of the foregoing items, further comprising functional biopeptides attached to the fibers.
123. The culture of any one of the foregoing items, further comprising functional biopeptides attached to the fibers.
124. The method of any one of the foregoing items, further comprising functional biopeptides attached to the fibers.
125. The biocompatible scaffold of any one of the foregoing items, further comprising functional biopeptides attached to the fibers.
126. The composition of any one of the foregoing items, wherein the fibers are coated with one or more of fibronectin, vitronectin and collagen.
127. The culture of any one of the foregoing items, wherein the fibers are coated with one or more of fibronectin, vitronectin and collagen.
128. The method of any one of the foregoing items, wherein the fibers are coated with one or more of fibronectin, vitronectin and collagen.
129. The biocompatible scaffold of any one of the foregoing items, wherein the fibers are coated with one or more of fibronectin, vitronectin and collagen.
130. The composition of any one of the foregoing items, wherein the fibers are coated with silica or graphene oxide.
131. The culture of any one of the foregoing items, wherein the fibers are coated with silica or graphene oxide.
132. The method of any one of the foregoing items, wherein the fibers are coated with silica or graphene oxide.
133. The biocompatible scaffold of any one of the foregoing items, wherein the fibers are coated with silica or graphene oxide.
134. The composition of any one of the foregoing items, further comprising one or more growth factors.
135. The culture of any one of the foregoing items, further comprising one or more growth factors.
136. The method of any one of the foregoing items, further comprising one or more growth factors.
137. The biocompatible scaffold of any one of the foregoing items, further comprising one or more growth factors.
138. The composition of any one of the foregoing items, wherein the growth factors are fibroblast growth factor (FGF), epidermal growth factor (EGF), transforming growth factor or platelet-derived growth factor (PDGF).
139. The culture of any one of the foregoing items, wherein the growth factors are fibroblast growth factor (FGF), epidermal growth factor (EGF), transforming growth factor or platelet-derived growth factor (PDGF).
140. The method of any one of the foregoing items, wherein the growth factors are fibroblast growth factor (FGF), epidermal growth factor (EGF), transforming growth factor or platelet-derived growth factor (PDGF).
141. The biocompatible scaffold of any one of the foregoing items, wherein the growth factors are fibroblast growth factor (FGF), epidermal growth factor (EGF), transforming growth factor or platelet-derived growth factor (PDGF).
142. The biocompatible scaffold of any one of the foregoing items, wherein the scaffold is 3D printed.
143. The method of any one of the foregoing items, wherein the scaffold is 3D printed.
144. A method for storing the biocompatible scaffold of any of the foregoing items, comprising washing the scaffold with phosphate buffered saline (PBS), optionally decellularizing the scaffold, and storing the scaffold in PBS.
145. The composition of any one of the foregoing items, wherein the PNPCs have two or more of the following characteristics: telomerase activity with extended replicative capacity (e.g., 40 cell doublings or more), normal karyotype, CD34⁻, CD45⁻, CXCL5⁺, PTGSI⁺, ANGPTL4⁺, low or no HLAII, CD90⁺, CD49c.
146. The culture of any one of the foregoing items, wherein the PNPCs have two or more of the following characteristics: telomerase activity with extended replicative capacity (e.g., 40 cell doublings or more), normal karyotype, CD34⁻, CD45⁻, CXCL5⁺, PTGSI⁺, ANGPTL4⁺, low or no HLAII, CD90⁺, CD49c.
147. The method of any one of the foregoing items, wherein the PNPCs have two or more of the following characteristics: telomerase activity with extended replicative capacity (e.g., 40 cell doublings or more), normal karyotype, CD34⁻, CD45⁻, CXCL5⁺, PTGSI⁺, ANGPTL4⁺, low or no HLAII, CD90⁺, CD49c.
148. The biocompatible scaffold of any one of the foregoing items, wherein the PNPCs have two or more of the following characteristics: telomerase activity with extended replicative capacity (e.g., 40 cell doublings or more), normal karyotype, CD34⁻, CD45⁻, CXCL5⁺, PTGSI⁺, ANGPTL4⁺, low or no HLAII, CD90⁺, CD49c.

### Post Natal Progenitor Cells (PNPCs)

As described herein aspects of the invention relate to the administration of PNPCs to a subject to treat tissue damage.

Aspects of the invention as herein described provide methods of administering the cells to a subject having tissue damage, so as to have the beneficial effect of one or more but not necessarily any or all of preventing, ameliorating, inhibiting, or curing tissue damage. Cells and methods in accordance therewith are described below.

PNPCs in accordance with various embodiments of the invention can be isolated from a variety of compartments and tissues of such mammals in which they are found, including but not limited to, bone marrow, peripheral blood, cord blood, blood, spleen, liver, muscle, brain, adipose tissue, placenta and others discussed below. PNPCs in some embodiments are cultured before use.

In some embodiments PNPCs are isolated from bone marrow. In some particular embodiments in this regard, PNPCs are isolated from human bone marrow.

In many embodiments PNPCs are not genetically engineered.

In some embodiments PNPCs are genetically engineered. PNPCs can be genetically engineered for a wide variety of purposes, such as those well known to the art. For instance, they can be engineered to have improved growth characteristics, to improve their therapeutic efficacy, to express one or more exogenous genes to produce beneficial substance, and to alter their immunological profiles.

In some embodiments genetically engineered PNPCs are produced by in vitro culture. In some embodiments genetically engineered PNPCs are produced from a transgenic organism.

In many embodiments the purity of PNPCs on the scaffold or for administration to a subject is about 100%. In other embodiments it is 95% to 100%. In some embodiments it is 85% to 95%. Particularly in the case of admixtures with other cells, the percentage of PNPCs can be 2%-5%, 3%-7%, 5%-10%, 7%-15%, 10%-15%, 10%-20%, 15%-20%, 20%-25%, 25%-30%, 30%-35%, 35%-40%, 40%-45%, 45%-50%, 50-60%, 60%-70%, 70%-80%, 80%-90%, or 90%-95%.

In some embodiments the purity of the cells for administration is about 100% (substantially homogeneous). In other embodiments it is 95% to 100%. In some embodiments it is 85% to 95%. Particularly, in the case of admixtures with other cells, the percentage can be about 10%-15%, 15%-20%, 20%-25%, 25%-30%, 30%-35%, 35%-40%, 40%-45%, 45%-50%, 50-60%, 60%-70%, 70%-80%, 80%-90%, or 90%-95%. Or isolation/purity can be expressed in terms of cell doublings where the cells have undergone, for example, 10-20, 20-30, 30-40, 40-50 or more cell doublings.

Treatment of disorders or diseases or the like with PNPCs may be with non-induced PNPCs. Treatment also may be with PNPCs that have been induced so that they are committed to a differentiation pathway. Treatment also may involve PNPCs that have been induced to differentiate into a less potent stem cell with limited differentiation potential. It also may involve PNPCs that have been induced to differentiate into a terminally differentiated cell type. The best type or mixture of PNPCs will be determined by the particular circumstances of their use, and it will be a matter of routine design for those skilled in the art to determine an effective type or combination of PNPCs in this regard.

The cells can naturally achieve these effects (i.e., when they are not genetically or pharmaceutically modified). However, the cells also can be genetically or pharmaceutically modified to increase effectiveness and/or improve their properties.

However, cells may be treated with one or more agents that will induce or increase the effect. Thus, substantially homogeneous populations may be treated to enhance the effect.

If the population is not substantially homogeneous, then, it is preferable that the parental cell population to be treated contains at least 100 of the desired cell type in which enhanced effect is sought, more preferably at least 1,000 of the cells, and still more preferably, at least 10,000 of the cells. Following treatment, this sub-population can be recovered from the heterogeneous population by known cell selection techniques and further expanded if desired.

In one embodiment, the PNPCs have undergone a desired number of cell doublings in culture. For example, the cells have undergone at least 10-40 cell doublings in culture, such as 30-35 cell doublings or more (e.g., >40), and wherein the cells are not transformed and have a normal karyotype. If cells are transformed or tumorigenic, and it is desirable to use them for infusion, such cells may be disabled so they cannot form tumors in vivo, as by treatment that prevents cell proliferation into tumors. Such treatments are well known in the art.

### Multipotent Adult Progenitor Cells (MAPCs)

Effective atmospheric oxygen concentrations of less than about 10%, including about 3 to 5%, can be used at any time during the isolation, growth, and differentiation of MAPCs in culture.

In additional experiments, the density at which MAPCs are cultured can vary from about 100 cells/cm² or about 150 cells/cm² to about 10,000 cells/cm², including about 200 cells/cm² to about 1500 cells/cm² to about 2000 cells/cm². The density can vary between species. Additionally, optimal density can vary depending on culture conditions and source of cells. It is within the skill of the ordinary artisan to determine the optimal density for a given set of culture conditions and cells.

Cells may be cultured under various serum concentrations, e.g., from 0-20%, particularly 15-20%. When serum is included, fetal bovine serum may be used. Higher serum may be used in combination with lower oxygen tensions, for example, about 15-20% serum with 3-5% oxygen. In a preferred embodiment, serum-free medium is used, and can be supplemented with one or more growth factors. When propagating cells for expansion prior to use in accordance with the present invention, cells need not be selected prior to adherence to culture dishes. For example, after a Ficoll gradient, cells can be directly plated, e.g., 250,000-500,000/cm². Adherent colonies can be picked, possibly pooled, and further expanded.

In one embodiment, high serum (around 15-20%) and low oxygen (around 3-5%) conditions are used for the cell culture. Specifically, adherent cells from colonies are plated and passaged at densities of about 1700-2300 cells/cm² in high serum and low oxygen (with PDGF and EGF).

### Seeding

### Culturing Conditions

Seeding and expanding multipotent adult progenitor cells, allowing the cells to deposit extracellular matrix (ECM) on the electrospun fibers, optionally decellularizing and analyzing the deposited ECM and optionally reseeding of fresh cells on the ECM construct can be performed by various methods and in various incubators.

Any medium can be used to culture the cells, but parameters should be examined in order to maintain the undifferentiated state of the PNPCs, or alternatively to allow for differentiation if desired. The medium can be supplemented with fetal bovine serum (FBS) or fetal calf serum (FCS) for growth conditions, however, serum-free medium may preferably be used and can be supplemented with certain growth factors, including epidermal growth factor (EGF), platelet-derived growth factor (PDGF) and fibroblast growth factor (FGF). Certain additives may be included such as glucose, antibiotics such as gentamycin or penicillin/streptomycin.

Cells are typically incubated at 37° C.; 5.5% CO₂ and low O₂, but these conditions can be varied slightly and still maintain viable cells.

Growth of cells can be assessed by any means known in the art. Cells can be identified by staining, for example calcein staining. Cell growth can also be assessed by measuring their consumption of nutrients, such as glucose, the production of by-products, such as lactate, the production of extracellular matrix components such as fibronectin and procollagen, and the production of growth factors, such as CXCL5, IL-8, and VEGF. Cell growth can also be assessed by determining the DNA content of the cell culture.

### Expansion

If expansion of cells is desired prior to use on the scaffolds in accordance with the present invention, expansion of cells may be performed as in the Examples, discussed below in Example 1. Minor variations in culture conditions are envisioned. Once cells approach confluence, they are removed from the plate or flask using trypsin/EDTA and seeded at desired density ranging between 500-2500 cells per cm², preferably about 2000 cells/cm².

### Treatment Using PNPCs

Doses (i.e., the number of cells) for humans or other mammals can be determined without undue experimentation by the skilled artisan, from this disclosure, the documents cited herein, and the knowledge in the art. The optimal dose to be used in accordance with various embodiments of the invention will depend on numerous factors, including the following: the disease being treated and its stage; the species of the donor, their health, gender, age, weight, and metabolic rate; the donor's immunocompetence; other therapies being administered; and expected potential complications from the donor's history or genotype. The parameters may also include: whether the cells are syngeneic, autologous, allogeneic, or xenogeneic; their potency; the site and/or distribution that must be targeted; and such characteristics of the site such as accessibility to cells. Additional parameters include coadministration with other factors (such as growth factors and cytokines). The optimal dose in a given situation also will take into consideration the way in which the cells are formulated, the way they are administered (e.g., perfusion, intra-organ, etc.), and the degree to which the cells will be localized at the target sites following administration.

The invention is also directed to cell populations with specific potencies for achieving any of the effects described herein. As described above, these populations are established by selecting for cells that have desired potency. These populations are used to make other compositions, for example, a cell bank comprising populations with specific desired potencies and pharmaceutical compositions containing a cell population with a specific desired potency.

### Scaffolds

Scaffolds can be prepared by any method, including electrospinning and 3D printing.

### Electrospinning

In one embodiment, scaffolds are prepared by electrospinning fibers into a scaffold. Exemplary methods for electrospinning are described in U.S. Pat. No. 9,766,228, which is incorporated herein by reference in its entirety.

Factors to be considered in choosing an appropriate electrospun fiber include the material from which the fiber is produced. The fiber may be composed of any material, including natural materials, such as alginate, cellulose, chitin, chitosan, hydroxyapatite, hyaluronic acid, starch, dextran, heparin, silk, gelatin, keratin or fibrinogen. Alternatively, the fiber may be composed of a synthetic polymer, such as poly(α-hydroxy acids) such as polyhydroxy alkanoate (PHA), polydroxybutyrate (PHB), poly(3-hydroxybutyrate-co-3-hydroxyvalerate) (PHBV), poly lactic (PLA) or glycolic acids such as poly(lactic acid-co-glycolic acid) (PLGA), poly(ε-caprolactone) (PCL), polyurethane (PU), polyphosphazene, polystyrene (PS), polyacrylonitrile (PAN), polycarbonate (PC), polyvinylpyrrolidone (PVP), polybutadiene, polyvinylbutyral (PVB), polyvinyl chloride (PVC), polyvinyl methyl ether (PVME), polyester, poly ethylene oxide (PEO), polyaniline (PANI), polyfluorenes, polypyrroles (PPY), poly ethylene dioxythiophene (PEDOT), polyphosphazenes, poly(propylene carbonate), poly(vinyl alcohol) (PVA), poly acrylo nitrile (PAN), poly vinyl methyl ether (PVME) or a mixture of two or more of those. Alternatively, the fibers may be supramolecular, such as multi-walled carbon nanotubes.

Another factor be considered is the diameter of the fiber. The fibers may have a diameter of about 5 nm-100 μm, preferably about 500 nm-50 μm, more preferably about 750 nm-25 μm, and most preferably about 10 μm. Diameter of the fiber can be controlled by altering the solutions used for electrospinning, as disclosed in U.S. Pat. No. 9,766,228, as well as the particular syringe/cannula used for extruding the fiber, the concentration of the polymer employed, pH, temperature, salt, solvent and solvent ratios, humidity, feeding rate, voltage, conductivity, and distance from the nozzle tip to the collector.

Another factor to be considered is the roughness of the fiber. Fibers are coated to increase roughness. The fibers may be coated with poly(ethylene oxide terephthalate)/poly(butylene terephthalate), oxygen and argon to alter their roughness. The fibers may be chosen based on a particular roughness value (Rₐ).

Yet another factor to be considered is the porosity (air to fiber volume) of the fiber. The porosity may be in the range of 60-95% open spaces, preferably 65-90%, more preferably 70-90%, and most preferably 75-85% open spaces.

Another factor to be considered is whether the fibers will be coated with a bioactive substance to enhance cell or protein adhesion to the fiber. The fibers may be coated with any material to alter their adhesion to the fiber. One such material is a functional biopeptide, such as fibronectin, vitronectin and collagen. The fibers may be coated with silica or graphene oxide or with one or more growth factors, such as fibroblast growth factor (FGF), epidermal growth factor (EGF), transforming growth factor or platelet-derived growth factor (PDGF).

Yet another factor to be considered is how the fibers are aligned. The fibers may be randomly oriented, aligned in parallel, cross-aligned, semi-aligned or perpendicular, or any combination thereof.

### 3D scaffolds

The preparation of 3D printed scaffolds for cell culture is described in various patents, including U.S. Pat. Nos. 10,213,967, 10,197,563, 10,105,392, 9,506,907 and 9,220,810, which are incorporated by reference herein in their entireties. For example, U.S. Pat. No. 10,213,967 describes a process for preparing a 3D scaffold, comprising the following steps: (A) supplying a gel solution and an airflow into a bubble generator to form a plurality of bubbles; (B) supplying the bubbles into a bubble mixing channel through which the bubbles flow to a bubble collector; (C) adding a coagulating solution into the bubble mixing channel before the bubbles are collected to result in a gel coagulation effect in the bubble mixing channel; (D) collecting the bubbles in the bubble collector before the gel coagulation effect is finished; wherein the gel coagulation effect is a reaction that a foam containing the gel solution is coagulated into a solid-state structure; and (E) communicating with at least a part of the bubbles to form a 3D scaffold, wherein the bubble mixing channel is connected to a coagulating solution channel through which the coagulating solution is added, and the bubble mixing channel includes at least a bent portion and a first outlet, the bent portion is disposed between the first outlet and an intersection of the bubble mixing channel and the coagulating solution channel, the bubbles start to contact the coagulating solution at the intersection of the bubble mixing channel and the coagulating solution channel and the gel coagulation effect is thus started; wherein the bubbles are changed in shape because of flowing through the bent portion.

One variable to be considered when generating a 3D scaffold is the role of porosity and pore size, as described in Loh et al., Tissue Eng. Part B. Rev. 19(6):485-502 (December 2013), which is incorporated herein by reference. Salt leaching is one method by which a 3-D scaffold can be produced, wherein salt is placed in a mold and then a polymer is poured in and the salt removed to create a hardened polymore with pores. Alternatively, gas can be used as a porogen, using solid discs of polymers such as polyglycoline and poly-L-lactide, through which high pressure carbon dioxide is applied. This method eliminates the need for harsh chemical solvents. Another method is phase separation, in which a polymer is dissolved in a suitable solvent, placed in a mold, and rapidly cooled to freeze the solvent. One other method is freeze-drying.

3D printing can also be used to create a scaffold, by laying down successive layers of material (e.g., a powder) using an “inkjet” print head. Advantages of 3D printing are enabling better control of pore sizes, pore morphology and porosity of matrix, as well as high resolution and controlled internal structures. 3D printing techniques can be categorized into powder-based 3D printing, ink-based 3D printing, and polymerization-based printing. Structures are first modeled using UG, CATIA, ProE or other customized software. Then an ST-format file containing all the model information is exported to the 3D printing system to construct the scaffold layer-by-layer.

Materials to be used for 3D printing may have characteristics including biocompatibility, bioactivity, biodegradability and non-immunogenicity. Exemplary materials for creating the 3D printed scaffold include poly(lactic acid) (PLA), polycaprolactone (PCL), poly(glycolic acid) (PGA) or their copolymers. Bioactive hard phase materials may also be included, such as non-degradable bio-ceramics such as alumina and zirconia, and bioactive glasses. Bioinks may include alginate, chitosan, agarose, hyaluronic-MA, fibrin, silk fibroin, gelatin, collagen type 1, decellularized ECM, Matrigel, methylcellulose, poly(ethylene glycol)poly(ethylene oxide) and pluronic F127, among other materials disclosed herein.

An additional factor to be considered is the mechanical property of the scaffold, which should be tailored for the specific site at which it is to be implanted. Such properties include compressive strength, elastic stiffness, fracture toughness and relaxation.

Optimal pore size may be determined by a person of ordinary skill in the art based on the desired application, but may be from about 20-1000 μm, preferably between about 200-500 μm, most preferably between 250-450 μm, with a porosity between about 50 and about 90%, more preferably between about 60 and about 80%.

### Sterilization and Processing

Sterilization and processing of electrospun fibers can be performed by various processes and equipment. Typical temperatures for sterilization range from about 100-200° C., more preferably about 120-170° C. Typical times for sterilization range from about 20 minutes to about 3 hours, more preferably about 60-150 minutes. Scaffolds can be sterilized using steam (autoclave), wherein two common sterilization settings are temperatures of 121° C. at 30 minutes or 132° C. at 4 minutes in prevacuum sterilizer. Dry heat can be used, for example 170° C. for 60 minutes, 160° C. for 120 minutes and 150° C. for 150 minutes, ethanol (e.g., 70%), peracetic acid, UV (wavelength 240-280 nm), electron beam (e.g., 50-300 kGy) or gamma radiation (e.g., 25-65 kGy) or ethylene oxide gas (e.g., at about 37 to 63° C., relative humidity of 40 to 80% and temperature of 37 to 63° C.). Various methods of sterilizing are disclosed in Valente et al. (2016) ACS AppL Mater. Interfaces 8(5):3241-9, which is incorporated by reference herein in its entirety.

### ECM Deposition

### Optional Decellularization

When the PNPCs adhere and are cultured on the scaffold, they deposit extracellular matrix (ECM). The PNPCs can then optionally be removed (“decellularization”), leaving the ECM for further culturing of PNPCs, or alternatively, other types of cells what are desired for administration. Cells can be removed by any known method, including but not limited to treatment with various detergents such as Triton-X 100 or through mechanical means, or by sonication. The use of detergents is preferred.

### ECM Analysis

Once deposited, the ECM is analyzed for content, such as by using PicroSirius red to stain for total collagen. Alternatively, or in addition, ECM mRNA expression can be assessed. For example, COL1A1 expression (gene for type I collagen), COL3A1 expression (gene for type III collagen), COL10A1 expression (gene for alpha chain of type X collagen), COLA2 expression (gene for type I collagen, alpha 2 chain), DCN expression (gene for decorin), FN expression (gene for fibronectin), DPT expression (gene for dermatopontin), and/or LOX expression (gene for lysyl oxidase) can be assessed.

### Reseeding

Once the ECM has been deposited on the scaffold, PNPCs can be reseeded onto the scaffold using methods described above and in the Examples below. Alternatively, PNPCs can be induced to differentiate into other cell types for transplantation, and culture conditions suitable for inducing differentiation into particular cell types are known in the art.

Other cell types may either be combined with the PNPCs, or used instead of PNPCs to adhere to the ECM on the scaffold. Examples of cell types that might be used in combination with PNPCs include but are not limited to endothelial, epithelial, fat, bone, muscle, tendon, cartilage, neurological, immunologic, pancreatic cells. Examples of cell types that might be used instead of PNPCs include endothelial, epithelial, fat, bone, muscle, tendon, cartilage, neurological, immunologic, pancreatic cells.

### Methods of Treatment

The cultures and compositions of the present invention can be used to treat any disease or condition where cell growth or tissue repair is desired. Examples of such conditions include bone injury or bone loss, blood disorders, diseases of the muscle, spinal cord injury, brain injury, neurodegenerative disease, heart and vasculature disease, liver disease, diabetes, disease of the intestine and colon, and repair of tissue damage caused by burns or injury or as a result of or for tissue grafts during surgery.

The following examples are provided to aid the understanding of the present invention, the true scope of which is set forth in the appended claims. It is understood that modifications can be made in the procedures set forth without departing from the spirit of the invention.

### EXAMPLES

The compositions and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the processes, formulations and/or methods of the invention may be made without departing from the spirit of the invention and the scope of the appended claims.

### Example 1

### Production of Multipotent Adult Progenitor Cells (MAPC)

Human MAPCs as used in the example were isolated from a single bone marrow aspirate, purchased from Lonza (Walkersville, MD). The bone marrow was diluted with Phosphate Buffered Saline (PBS), and cell fractions were separated using Histopaque-1077. The mononuclear cell fraction was washed with PBS and seeded at a density of 2400 cells/cm² on Fibronectin (FN, Sigma, 6.7 ng per cm²) coated plastic flasks in MAPC culture medium (60% Dulbecco's modified Eagle's medium (DMEM) 1 g/l glucose without L-glutamine (Lonza) supplemented with high fetal bovine serum (FBS; Atlas Biologicals, Fort Collins, CO), 1× insulin-transferrin selenium liquid medium supplement (Lonza), 40% MCDB-201 (Sigma-Aldrich), 10 ng/ml platelet-derived growth factor and 10 ng/ml epidermal growth factor (R&D Systems, Minneapolis, MN), 50 nM dexamethasone (Sigma-Aldrich), 100 U/ml penicillin/streptomycin (Lonza), 10⁻⁴ M 2-phospho-L-ascorbic acid (Sigma), and 0.5× linoleic acid—albumin (Sigma-Aldrich)), and incubated at 37° C. in a humidified atmosphere of 5% CO₂ and low O₂.

After 8 days, when clones were formed, cells were lifted using Trypsin EDTA (0.25%, Lonza], counted and seeded at 920 cells per cm² in a FN coated T-flask. Based on the starting cell numbers, the Population Doubling (PD) at this stage was calculated as PD=14.29.

From this stage, cells were allowed to grow for 2 or 3 days and subcultured before reaching confluence. After detachment by TrypLE, cells were counted and seeded in new FN coated flasks at density of 2000 cells/cm². Population doublings were calculated based on the number of cells initially seeded (Ci) and the number of cells harvested (Ch) using the following equation: PDh=PDi+log₂(Ch/Ci).

Cells were cultured until PD 24.2 and cryopreserved in PlasmaLyte (Baxter) supplemented with 5% human Serum Albumin (CAF-DCF, Belgium) and 10% dimethyl sulfoxide (Sigma).

Prior to the matrix deposition experiment in the example, cells were thawed and plated on a tissue culture flask in MAPC medium at a density of 2000 cells per cm² and allowed to grow for 2 days at 37° C. in a humidified atmosphere of 5% CO₂ and low O₂.

### Example 2

### Optimization of Scaffold

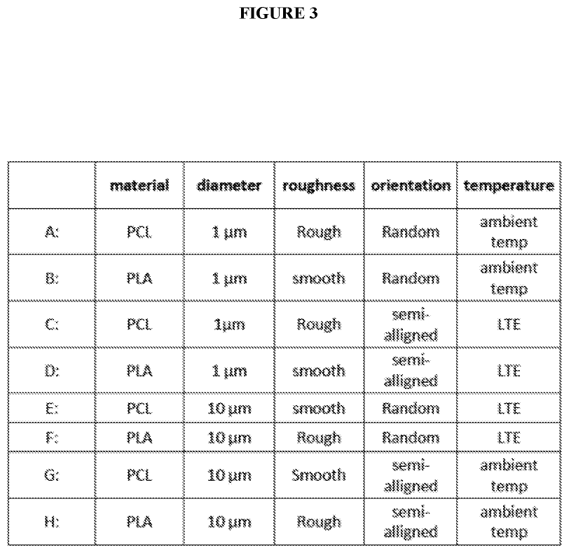

Various materials were tested for their ability to sustain MAPC. Materials tested included Ti6AI4V diamond scaffolds, PLA discs/cubes, Visijet® 3D printed scaffolds and PLA/PCL electrospun sheets. Various parameters of electrospun sheets were analyzed, including PCL vs. PLA, diameter of 1 μm vs. 10 μm, smooth vs. rough, random vs. semi-aligned fiber orientation and ambient temperature vs. ultra-low temperature. FIG. 3. With respect to electrospun scaffolds tested as in FIG. 3, conclusions were that 10 μm fiber diameter results in better proliferation and ECM production than the 1 μm fiber diameter. When looking at the 10 μm diameter: (1) there is no pronounced effect of the LTE vs. ambient temperature; (2) PLA seems to result in a slightly better proliferation and ECM production than PCL; (3) semi-aligned fiber structures seem to perform better than the random structures; (4) structural integrity of the random aligned sheets is higher and therefore more applicable in a clinical setting; and (5) a rough surface also seems to perform a bit better than a smooth surface. As such, PLA, 10 μm diameter, random alignment, rough surface and ambient temperature were chosen. However, it should be noted that the surgeon found PCL to be preferable when handling the material.

### Example 3

### Sterilization of Scaffolds

The production process was optimized at the steps of: (1) sterilization; (2) surface functionalization; (3) seeding/culture; (4) decellularization; and (5) reseeding.

For sterilization, sheets of electrospun material and CellCrown™ suspension aids were submerged in 70% ethanol for 90 minutes, ethanol was removed and the sheets were left to dry overnight in a laminar flow hood. Alternatively, sterilization may be by peracetic acid, gamma radiation or ethylene oxide gas.

Poly(ε-caprolactone) (PCL) fibers 10 μm thick were spun into randomly aligned sheets 300-350 μm thick. Sheets were placed in 12-well CellCrown™ suspension aids and the sheets and plates were treated with 70% ethanol for 90 minutes and dried overnight in a laminar flow cabinet.

### Example 4

### Production of Extracellular Matrix (ECM)

10,000 MAPCs were seeded onto each sheet produced, sterilized and activated (as in Examples 1-5) in 100 μl culture medium (above) containing recombinant human epidermal growth factor and recombinant human platelet-derived growth factor with antibiotic. Cells were incubated for 1 hour at 37° C. in 5.5% CO₂ and low O₂. 100 μl of medium was added to each sheet and cells were further incubated for 1 hour at 37° C. in 5.5% CO₂ and 5% O₂. Subsequently, 3 ml of medium was added to each sheet and incubated at 37° C. in 5.5% CO₂ and low O₂ for 2 weeks with glucose/lactate measurements every 2-3 days. Cells were cultured for 2 weeks to produce extracellular matrix. Medium was refreshed according to lactate production and glucose consumption. Medium was exchanged every 2-3 days, starting with 3 ml/sheet. When the glucose level dropped to around 0.3 g/l, the volume of medium was increased (to about 20 ml after 2 weeks). The sheets were also transferred to 50 ml tubes to hold this volume.

### Example 5

### Decellularization and Storage of ECM Sheets

Sheets produced in Example 1 were washed twice with phosphate buffered saline (PBS). 3 ml of decellularization solution containing triton X-100 was added and the scaffold/cells were incubated for 10 minutes at 37° C. on a moving platform. The decellularization solution was removed and the sheets were washed three times in PBS and stored in 3 ml PBS and penicillin-streptomycin at 2-8° C. until further use. If the scaffold is to be used for reseeding immediately, it is washed one time in culture medium prior to adding cells. ECM can be detected with PicroSirius Red at various time points.

### Example 6

### Reseeding of ECM

Decellularized sheets were washed thoroughly in PBS and put in growth medium while cells are harvested for reseeding. Sheets were reseeded by incubating 100,000 cells per sheet in 50 μl growth medium for 1 hour at 37° C. in 5.5% CO₂ and low O₂. An additional 50 μl growth medium was added and incubated for 1 hour at 37° C. in 5.5% CO₂ and low O₂. 3 ml growth medium was added to each sheet, and the sheets were cultured at 37° C.; 5.5% CO₂ and low O₂ for 2-3 weeks, refreshing the medium three times per week.

### Example 7

### Cryopreservation of ECM and Reseeded ECM

Sheets with deposited ECM or with ECM and reseeded cells were placed in 12-well plates with medium. 3 ml of PlasmaLyte with 5% HSA without dimethylsulfoxide (DMSO) was added to the sheets and they were cooled to 2-8° C. in the refrigerator. The medium was removed and 3 ml of medium with 10% DMSO (cryopreservant) was added to each sheet. The 12-well CellCrown™ suspension aids containing the sheets were covered with parafilm and transferred to a styrofoam box with filling and stored overnight at −80° C. overnight. Plates were transferred to the gas phase of a liquid nitrogen tank for longer storage.

### Example 8

### Thawing of Cryopreserved Sheets

The 12-well CellCrown™ suspension aids were removed from the liquid nitrogen tank and placed in an incubator at 37° C. in 5.5% CO₂ and low O₂ until the sheets were thawed (up to 90 minutes). The cryopreservant was removed and replaced with 3 ml growth medium.

### Example 9

### Visualizing Cells on ECM

Cells were washed with PBS. Staining solution (PBS+1.85 μM calcein) was added and cells were stained for 20 minutes at 37° C. The stain was removed and the sheets were washed with PBS and visualized with the SYNENTEC Cellavista cell imaging system at 470 nm.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

### REFERENCES

Amani, H., W. R. Dougherty, and S. Blome-Eberwein, Use of Transcyte and dermabrasion to treat burns reduces length of stay in burns of all size and etiology. Burns, 2006. 32(7): p. 828-32.

Apligraf. (www.apligaf.com/professional/clinical_data/DFU/efficacy.html), consulted on Jan. 13, 2015 Online.

Aranguren, X. L., et al., In vitro and in vivo arterial differentiation of human multipotent adult progenitor cells. Blood, 2007. 109(6): p. 2634-42.

Aranguren et al. 2011. MAPC transplantation confers a more durable benefit than AC133+cell transplantation in severe hind limb ischemia. Cell Transplant. 20(2): 259-269

Armstrong, D. G., L. A. Lavery, and C. Diabetic Foot Study, Negative pressure wound therapy after partial diabetic foot amputation: a multicentre, randomised controlled trial. Lancet, 2005. 366(9498): p. 1704-10.

Badylak, S. F., D. O. Freytes, and T. W. Gilbert, Extracellular matrix as a biological scaffold material: Structure and function. Acta Biomater, 2009. 5(1): p. 1-13.

Balaji, S., S. G. Keswani, and T. M. Crombleholme, The Role of Mesenchymal Stem Cells in the Regenerative Wound Healing Phenotype. Adv Wound Care (New Rochelle), 2012. 1(4): p. 159-165.

Beerens et al. 2018. Multipotent Adult Progenitor Cells Support Lymphatic Regeneration at Multiple Anatomical Levels during Wound Healing and Lymphedema. Sci Rep 8(1):3852

Bello, Y. M., A. F. Falabella, and W. H. Eaglstein, Tissue-engineered skin. Current status in wound healing. Am J Clin Dermatol, 2001. 2(5): p. 305-13.

Blume, P. A., et al., Comparison of negative pressure wound therapy using vacuum-assisted closure with advanced moist wound therapy in the treatment of diabetic foot ulcers: a multicenter randomized controlled trial. Diabetes Care, 2008. 31(4): p. 631-6.

Brown, B. N., et al., Surface characterization of extracellular matrix scaffolds. Biomaterials, 2010. 31(3): p. 428-37.

Carsin, H., et al., Cultured epithelial autografts in extensive burn coverage of severely traumatized patients: a five year single-center experience with 30 patients. Burns, 2000. 26(4): p. 379-87.

Chen, L., et al., Paracrine factors of mesenchymal stem cells recruit macrophages and endothelial lineage cells and enhance wound healing. PLoS One, 2008. 3(4): p. e1886.

Chen, L., et al., Analysis of allogenicity of mesenchymal stem cells in engraftment and wound healing in mice. PLoS One, 2009. 4(9): p. e7119.

Cheng, H. W., et al., Decellularization of chondrocyte-encapsulated collagen microspheres: a three-dimensional model to study the effects of acellular matrix on stem cell fate. Tissue Eng Part C Methods, 2009. 15(4): p. 697-706.

Crabbé et al. 2016. Using miRNA-mRNA Interaction Analyses to Link Biologically Relevant miRNAs to Stem Cell Identity Testing for Nex-Generation Development. Stem Cells Transl Med 5(6):709-722

Cullen, B., et al., Mechanism of action of PROMOGRAN, a protease modulating matrix, for the treatment of diabetic foot ulcers. Wound Repair Regen, 2002. 10(1): p. 16-25.

Cunha et al. 2017. Human multipotent adult progenitor cells enhance islet function and revacularisation when co-transplanted as a composite pellet in a mouse model of diabetes.Diabetologia 60:134-142

Curran, M. P. and G. L. Plosker, Bilayered bioengineered skin substitute (Apligraf): a review of its use in the treatment of venous leg ulcers and diabetic foot ulcers. BioDrugs, 2002. 16(6): p. 439-55.

Egana, J. T., et al., Use of human mesenchymal cells to improve vascularization in a mouse model for scaffold-based dermal regeneration. Tissue Eng Part A, 2009. 15(5): p. 1191-200.

Epicel. (http://www.epicel.com/hcp/about/epicel-made.aspx), consulted on Feb. 9, 2015 Online.
Falanga, V. and M. Sabolinski, A bilayered living skin construct (APLIGRAF) accelerates complete closure of hard-to-heal venous ulcers. Wound Repair Regen, 1999. 7(4): p. 201-7.

Gallagher, J., et al., Dynamic Tensile properties of Human Skin. IRCOBI Conference 2012, 2012. IRC-12-59.

Gantwerker, E. A. and D. B. Horn, Skin: histology and physiology of wound healing. Clin Plast Surg, 2012. 39(1): p. 85-97.

Gebler, A., O. Zabel, and B. Seliger, The immunomodulatory capacity of mesenchymal stem cells. Trends Mol Med, 2012. 18(2): p. 128-34.

Ghatnekar, O., M. Willis, and U. Persson, Cost-effectiveness of treating deep diabetic foot ulcers with Promogran in four European countries. J Wound Care, 2002. 11(2): p. 70-4.

Gomathysankar, S., A. S. Halim, and N. S. Yaacob, Proliferation of keratinocytes induced by adipose-derived stem cells on a chitosan scaffold and its role in wound healing, a review. Arch Plast Surg, 2014. 41(5): p. 452-7.

Gottrup, F., et al., Randomized controlled trial on collagen/oxidized regenerated cellulose/silver treatment. Wound Repair Regen, 2013. 21(2): p. 216-25.

Gurtner, G. C., et al., Wound repair and regeneration. Nature, 2008. 453(7193): p. 314-21.

Guyot, Y., et al., A computational model for cell/ECM growth on 3D surfaces using the level set method: a bone tissue engineering case study. Biomech Model Mechanobiol, 2014. 13(6): p. 1361-71.

Hankin, C. S., et al., Clinical and cost efficacy of advanced wound care matrices for venous ulcers. J Manag Care Pharm, 2012. 18(5): p. 375-84.

Hansen, S. L., et al., Using skin replacement products to treat burns and wounds. Adv Skin Wound Care, 2001. 14(1): p. 37-44; quiz 45-6.

Harding, K., M. Sumner, and M. Cardinal, A prospective, multicentre, randomised controlled study of human fibroblast-derived dermal substitute (Dermagraft) in patients with venous leg ulcers. Int Wound J, 2013. 10(2): p. 132-7.

Hart, C. E., A. Loewen-Rodriguez, and J. Lessem, Dermagraft: Use in the Treatment of Chronic Wounds. Adv Wound Care (New Rochelle), 2012. 1(3): p. 138-141.

Heimbach, D. M., et al., Multicenter postapproval clinical trial of Integra dermal regeneration template for burn treatment. J Burn Care Rehabil, 2003. 24(1): p. 42-8.

Hess et al. 2017. Safety and efficacy of multipotent adult progenitor cells in acute ischaemic stroke (MASTERS): a randomized, double-blind, placebo-controlled, phase 2 trial. Lancet Neurol 16(5): 360-368

Hu, M. S., et al., Tissue engineering and regenerative repair in wound healing. Ann Biomed Eng, 2014. 42(7): p. 1494-507.

Jackson, W. M., L. J. Nesti, and R. S. Tuan, Concise review: clinical translation of wound healing therapies based on mesenchymal stem cells. Stem Cells Transl Med, 2012. 1(1): p. 44-50.

Javazon, E. H., et al., Enhanced epithelial gap closure and increased angiogenesis in wounds of diabetic mice treated with adult murine bone marrow stromal progenitor cells. Wound Repair Regen, 2007. 15(3): p. 350-9.

Jiang, Y. et al., Pluripotency of mesenchymal stem cells derived from adult marrow. Nature, 2002, 418, 41-9

Karl, T., P. K. Modic, and E. U. Voss, [Indications and results of v.a.C therapy treatments in vascular surgery13 state of the art in the treatment of chronic wounds]. Zentralbl Chir, 2004. 129 Suppl 1: p. S74-9.

Kocyildirim et al. 2017. The Use of GMP-Produced Bone Marrow-Derived Stem Cells in Combination with Extracorporeal Membrane Oxygenation in ARDS: An Animal Model. ASAIO J 63(3): 324-332

Koutinas, M., et al., Bioprocess systems engineering: transferring traditional process engineering principles to industrial biotechnology. Comput Struct Biotechnol J, 2012. 3: p. e201210022.

Langer, A. and W. Rogowski, Systematic review of economic evaluations of human cell-derived wound care products for the treatment of venous leg and diabetic foot ulcers. BMC Health Serv Res, 2009. 9: p. 115.

Leung, A., T. M. Crombleholme, and S. G. Keswani, Fetal wound healing: implications for minimal scar formation. Curr Opin Pediatr, 2012. 24(3): p. 371-8.

LoGuidice et al. 2016. Multipotent adult progenitor cellson an allograft scaffold facilitate the bone repair process. J Tis Eng 7:1-14

Loh Q. L. et al., Three-dimensional Scaffolds for Tissue Engineering Applications: Role of Porosity and Pore Size, Tissue Eng Part B Rev, 2013, 19 (6), 485-502

Lovvorn, H. N., 3rd, et al., Relative distribution and crosslinking of collagen distinguish fetal from adult sheep wound repair. J Pediatr Surg, 1999. 34(1): p. 218-23.

Lu, H., et al., Cultured cell-derived extracellular matrix scaffolds for tissue engineering. Biomaterials, 2011. 32(36): p. 9658-66.

Ma, K., et al., Effects of nanofiber/stem cell composite on wound healing in acute full-thickness skin wounds. Tissue Eng Part A, 2011. 17(9-10): p. 1413-24.

Manne, J., et al., Collagen content in skin and internal organs of the tight skin mouse: an animal model of scleroderma. Biochem Res Int, 2013. 2013: p. 436053.

Mansilla, E., et al., Outstanding survival and regeneration process by the use of intelligent acellular dermal matrices and mesenchymal stem cells in a burn pig model. Transplant Proc, 2010. 42(10): p. 4275-8.

Margolis, D. J., et al., Diabetic neuropathic foot ulcers: the association of wound size, wound duration, and wound grade on healing. Diabetes Care, 2002. 25(10): p. 1835-9.
Margolis, D. J., et al., Prevalence of Diabetes, Diabetic Foot Ulcer, and Lower Extremity Amputation Among Medicare Beneficiaries, 2006 to 2008: Data Points #1, in Data Points Publication Series. 2011: Rockville (MD).

Marston, W. A., et al., The efficacy and safety of Dermagraft in improving the healing of chronic diabetic foot ulcers: results of a prospective randomized trial. Diabetes Care, 2003. 26(6): p. 1701-5.

Menendez-Menendez, Y., et al., Adult Stem Cell Therapy in Chronic Wound Healing. J Stem Cell Res Ther, 2014. 4(162).

Monami, M., et al., Autologous skin fibroblast and keratinocyte grafts in the treatment of chronic foot ulcers in aging type 2 diabetic patients. J Am Podiatr Med Assoc, 2011. 101(1): p. 55-8.

Natarajan, V., et al., Preparation and properties of tannic acid cross-linked collagen scaffold and its application in wound healing. J Biomed Mater Res B Appl Biomater, 2013. 101(4): p. 560-7.

O'Kane, S. and M. W. Ferguson, Transforming growth factor beta s and wound healing. Int J Biochem Cell Biol, 1997. 29(1): p. 63-78.

Pajardi, G., et al., Skin substitutes based on allogenic fibroblasts or keratinocytes for chronic wounds not responding to conventional therapy: a retrospective observational study. Int Wound J, 2014.

Park, S. A., et al., PDGF-BB does not accelerate healing in diabetic mice with splinted skin wounds. PLoS One, 2014. 9(8): p. e104447.

Peake, M. A., et al., Identification of a transcriptional signature for the wound healing continuum. Wound Repair Regen, 2014. 22(3): p. 399-405.

Pelacho et al. 2007. Multi potent adult progenitor cell transplantation increases vascularity and improves left ventricular function after myocardial infarction. J Tissue Eng Regen Med 1(1):51-59

Petersen, T. H., et al., Matrix composition and mechanics of decellularized lung scaffolds. Cells Tissues Organs, 2012. 195(3): p. 222-31.

Pittenger, M. F., et al., Multilineage potential of adult human mesenchymal stem cells. Science, 1999. 284(5411): p. 143-7.

Rojas et al. 2014. Human adult bone marrow-derived stem cells decrease severity of lipopolysaccharide-induced acute respiratory distress syndrome in sheep. Stem Cell Res Ther 5(2):42

Sen, C. K., et al., Human skin wounds: a major and snowballing threat to public health and the economy. Wound Repair Regen, 2009. 17(6): p. 763-71.

Shaw, J. E., R. A. Sicree, and P. Z. Zimmet, Global estimates of the prevalence of diabetes for 2010 and 2030. Diabetes Res Clin Pract, 2010. 87(1): p. 4-14.

Sheehan, P., et al., Percent change in wound area of diabetic foot ulcers over a 4-week period is a robust predictor of complete healing in a 12-week prospective trial. Diabetes Care, 2003. 26(6): p. 1879-82.

Singla, S., et al., Efficacy of topical application of beta urogastrone (recombinant human epidermal growth factor) in Wagner's Grade 1 and 2 diabetic foot ulcers: Comparative analysis of 50 patients. J Nat Sci Biol Med, 2014. 5(2): p. 273-7.

Snyder, R. J., et al., A post-hoc analysis of reduction in diabetic foot ulcer size at 4 weeks as a predictor of healing by 12 weeks. Ostomy Wound Manage, 2010. 56(3): p. 44-50.

Sohni, A. and C. M. Verfaillie, Multipotent adult progenitor cells. Best Pract Res Clin Haematol, 2011. 24(1): p. 3-11.

Taylor, B. M., Quantitative analysis of collagens in TheraSkin, ApliGraf and DermaGraft. Scientific Data Series, 2010. 20(02).

Tolar, J., et al., Host factors that impact the biodistribution and persistence of multipotent adult progenitor cells. Blood, 2006. 107(10): p. 4182-8.

Uccioli, L., et al., Two-step autologous grafting using HYAFF scaffolds in treating difficult diabetic foot ulcers: results of a multicenter, randomized controlled clinical trial with long-term follow-up. Int J Low Extrem Wounds, 2011. 10(2): p. 80-5.

Vaes, B., et al., Application of MultiStem((R)) Allogeneic Cells for Immunomodulatory Therapy: Clinical Progress and Pre-Clinical Challenges in Prophylaxis for Graft Versus Host Disease. Front Immunol, 2012. 3: p. 345.

Valente T A M., et al., Effect of Sterilization Methods on Electrospun Poly(lactic Acid) (PLA) Fiber Alignment for Biomedical Applications, 2016, ACS Appl Mater Interfaces, 8 (5), 3241-9

Van De Velde, K. and P. Kiekens, Biopolymers: Overview of several properties and consequences on their application. Polymer Testing, 2002. 21: p. 433-442.

Veves, A., P. Sheehan, and H. T. Pham, A randomized, controlled trial of Promogran (a collagen/oxidized regenerated cellulose dressing) vs standard treatment in the management of diabetic foot ulcers. Arch Surg, 2002. 137(7): p. 822-7.

Volk, S. W., S. A. Iqbal, and A. Bayat, Interactions of the Extracellular Matrix and Progenitor Cells in Cutaneous Wound Healing. Adv Wound Care (New Rochelle), 2013. 2(6): p. 261-272.

Vyas, K. S. and H. C. Vasconez, Wound healing: Biologics, Skin Substitutes, Biomembranes and Scaffolds. Healthcare, 2014. 2: p. 356-400.

Wainwright, D. J., Use of an acellular allograft dermal matrix (AlloDerm) in the management of full-thickness burns. Burns, 1995. 21(4): p. 243-8.

Walker, P. A., et al., Intravenous multipotent adult progenitor cell therapy for traumatic brain injury: preserving the blood brain barrier via an interaction with splenocytes. Exp Neurol, 2010. 225(2): p. 341-52.

Webster, J., et al., Negative pressure wound therapy for skin grafts and surgical wounds healing by primary intention. Cochrane Database Syst Rev, 2014. 10: p. CD009261.

Whitby, D. J. and M. W. Ferguson, Immunohistochemical localization of growth factors in fetal wound healing. Dev Biol, 1991. 147(1): p. 207-15.

White, R. and C. McIntosh, A review of the literature on topical therapies for diabetic foot ulcers. Part 2: Advanced treatments. J Wound Care, 2009. 18(8): p. 335-41.

Winters, C. L., et al., A multicenter study involving the use of a human acellular dermal regenerative tissue matrix for the treatment of diabetic lower extremity wounds. Adv Skin Wound Care, 2008. 21(8): p. 375-81.

Wolchok, J. C. and P. A. Tresco, The isolation of cell derived extracellular matrix constructs using sacrificial open-cell foams. Biomaterials, 2010. 31(36): p. 9595-603.

Wolchok, J. C. and P. A. Tresco, Using growth factor conditioning to modify the properties of human cell derived extracellular matrix. Biotechnol Prog, 2012. 28(6): p. 1581-7.

Wu, Y., et al., Mesenchymal stem cells enhance wound healing through differentiation and angiogenesis. Stem Cells, 2007. 25(10): p. 2648-59.

Xing, Q., et al., Decellularization of fibroblast cell sheets for natural extracellular matrix scaffold preparation. Tissue Eng Part C Methods, 2015. 21(1): p. 77-87.

Yang et al. 2017. Multipotent Adult Progenitor Cells Enhance Recovery After Stroke By Modulating the Immune Response from the Spleen. Stem Cells 35:1290-1302

Yeo, D., et al., Improving embryonic stem cell expansion through the combination of perfusion and Bioprocess model design. PLoS One, 2013. 8(12): p. e81728.

Zaulyanov, L. and R. S. Kirsner, A review of a bi-layered living cell treatment (Apligraf) in the treatment of venous leg ulcers and diabetic foot ulcers. Clin Intery Aging, 2007. 2(1): p. 93-8.

Zgheib, C., J. Xu, and K. W. Liechty, Targeting Inflammatory Cytokines and Extracellular Matrix Composition to Promote Wound Regeneration. Adv Wound Care (New Rochelle), 2014. 3(4): p. 344-355.

1. A composition comprising post-natal progenitor cells (PNPCs) seeded onto a 3D scaffold or a scaffold of fibers.

2. The composition of claim 1, wherein extracellular matrix from the PNPCs has been deposited on the 3D scaffold or scaffold of fibers.

3. The composition of claim 1, wherein the PNPCs are undifferentiated.

4. A culture of PNPCs, seeded onto the 3D scaffold or a scaffold of fibers according to claim 1.

5. The culture of claim 4, wherein the 3D scaffold of scaffold of fibers comprises extracellular matrix.

6. The culture of claim 4, wherein the PNPCs are undifferentiated.

7. A biocompatible scaffold prepared by: (a) seeding PNPCs onto a 3D scaffold or scaffold of fibers; and (b) allowing the PNPCs to deposit extracellular matrix onto the 3D scaffold or scaffold of fibers.

8. The biocompatible scaffold of claim 7, prepared with the additional step of: (c) decellularizing the scaffold.

9. The biocompatible scaffold of claim 8, prepared with the additional step of: (d) reseeding cells onto the scaffold to produce a re-cellularized scaffold.

10. The biocompatible scaffold of claim 9, wherein the reseeded cells are PNPCs.

11. A method for making the composition of claim 1, comprising seeding PNPCs onto a 3D scaffold or a scaffold of fibers.

12. The method for making a cell composition of claim 11, further comprising allowing the PNPCs to deposit extracellular matrix onto the 3D scaffold or scaffold of fibers.

13. (canceled)

14. The method for culturing cells of claim 12, wherein the 3D scaffold or scaffold of fibers comprises extracellular matrix deposited by the PNPCs.

15. A method for making the biocompatible scaffold of claim 7, comprising:(a) seeding PNPCs onto a 3D scaffold or a scaffold of fibers; and (b) allowing the cells to deposit extracellular matrix onto the 3D scaffold or scaffold of fibers.

16. The method for making a biocompatible scaffold of claim 15, comprising the further step of: (c) decellularizing the 3D scaffold or scaffold of fibers.

17. The method for making a biocompatible scaffold of claim 16, comprising the further step of: (d) reseeding cells onto the scaffold to produce a re-cellularized 3D scaffold or scaffold of fibers.

18. A method for treating a disease or condition in a patient, comprising administering to said patient the composition of claim 1.

19. A method for treating a disease or condition in a patient,comprising administering to said patient the composition of claim 2.

20. A method for treating a disease or condition in a patient, comprising administering to said patient the composition of claim 3.

21. A method for treating a disease or condition in a patient, comprising administering to said patient the biocompatible scaffold of claim 7.

22. (canceled)

23. (canceled)
